(19) United States
(12) Patent Application Publication
(10) Pub. No.: US 2020/0291286 A1
(43) Pub. Date: Sep. 17, 2020

(54) Emulsifiers for Invert Emulsion Drilling Fluids

(71) Applicant: Saudi Arabian Oil Company, Dhahran (SA)

(72) Inventors: Matthew Gary Hilfiger, Katy, TX (US); Carl Thaemlitz, Cypress, TX (US)

(21) Appl. No.: 16/888,119

(22) Filed: May 29, 2020

Related U.S. Application Data
(63) Continuation-in-part of application No. 15/867,027, filed on Jan. 10, 2018.
(60) Provisional application No. 62/444,883, filed on Jan. 11, 2017.

(51) Int. Cl. C09K 8/36; C07D 233/24

(52) U.S. Cl. CPC C09K 8/36; C07D 233/24

(57) ABSTRACT

The present disclosure relates to the creation of emulsifiers for use in an oil-based drilling mud. The disclosed emulsifiers may be created by the reaction of synthetic linear saturated fatty acids and polyamines under the conditions specified. The synthesized product may be used as a replacement for the wetting agent in conventional muds. Further improvements in conventional drilling muds are also disclosed where the synthesized product is used to replace incumbent wetting agents and the primary emulsifiers are replaced by synthetic fatty acids.

This application is a continuation-in-part of and claims priority from U.S. Non-Provisional application Ser. No. 15/867,027 filed Jan. 10, 2018, and titled “EMULSIFIERS FOR INVERT EMULSION DRILLING FLUIDS,” which claims priority from U.S. Provisional Application No. 62/444,883 filed Jan. 11, 2017, and titled “EMULSIFIERS FOR INVERT EMULSION DRILLING FLUIDS,” each of which are incorporated by reference in their entirety for purposes of United States patent practice.

### FIELD

The present disclosure generally relates to improved emulsifiers and additives for use in oil-based drilling mud systems.

### BACKGROUND

Drilling fluid, or drilling mud, is used to assist in the drilling of wells, including, for example, water, natural gas, or oil wells. These fluids are used for a variety of purposes including but not limited to applying hydrostatic pressure on the formation, keeping the drill bit from overheating, keeping the drill bit clean and lubricated, and carrying drill cuttings out of the wellbore.

Invert emulsion mud systems are oil-based muds where water is mixed with an oily substance such as diesel fuel. Due to the difficulties in mixing oil-based fluids with water, these systems are typically unstable. Stability is provided to the emulsion by the use of emulsifiers. Traditional emulsifiers for invert emulsion mud systems have relied on acid mixtures derived from tallow or tall oil sources. Tall oil is found in pine tree and is obtained as a by-product of the pulp and paper industry. As such, tall oil feedstocks are impure and require modification by the reaction of maleic anhydride, for example, in order to be used as an emulsifier. In addition, given the lack of pine trees, tall oil feedstocks are not widely available in the environments such as those found in the Middle East and other oil and gas producing regions.

### SUMMARY

An embodiment disclosed describes the synthesis of a wetting agent to be used in an oil-based mud. The wetting agent is synthesized from the reaction of a mixture of synthetic linear saturated fatty acids and a polyamine under the conditions described. The final product is a condensate comprising amidoamines and polyamide imidazolines.

The wetting agent product described previously may be used as a replacement for standard industry incumbent wetting agents in drilling mud formulations yielding improved performance over incumbent mud systems and is an additional embodiment.

In an additional embodiment, the wetting agent product may be further combined with a synthetic fatty acid, such as a mixture of C12-14 or C16-18, to yield a full emulsifier package replacement for drilling mud formulations which yields improved performance over incumbent systems.

An embodiment disclosed describes a process for creating a wetting agent for use in a non-aqueous drilling fluid to be used in an altered oil-based drilling mud. The process includes the steps of mixing a mixture of synthetic linear saturated fatty acid with a polyamine in the presence of an acid, for example, p-toluenesulfonic acid, heating the resulting mixture to a first temperature, for example, 160° C., for a first amount of time to create a first reaction product, then further heating the mixture to a second temperature, for example, 190° C., for a second amount of time to create a second reaction product. The mixture of synthetic linear saturated fatty acids in the process may have a carbon number of six to eighteen. The first amount of time may be four hours or time sufficient enough to drive off all water from the reaction. The second amount of time may be in the range of 2 to 3 hours. The first reaction product of the process may be a polyamide condensate. The second reaction product may be a mixture of polyamide imidazolines. In some embodiments, the second reaction product may also contain amidoamines. The polyamine in the reaction may consist of any one of or a combination of diethylenetriamine (DETA), triethylenetetramine (TETA), and tetraethylenepentamine (TEPA). The amount of p-toluenesulfonic acid may be 0.2% by weight. In some instances, the embodiment further comprises diluting the second reaction product with a solvent. The solvent used for dilution may consist of any one of or combination of xylene, ethylene glycol butyl ether, or n-butanol.

Another embodiment disclosed is an oil-based drilling mud that includes a non-aqueous drilling fluid and a wetting agent. The wetting agent is a mixture of polyamide imidazolines diluted with a solvent. The drilling fluid of this embodiment may be free of tall oil and tall oil products. The drilling fluid may contain a base oil, a viscosifier, water, calcium chloride, lime, an emulsifier, a fluid loss additive, barite, and calcium carbonite.

An additional embodiment disclosed is an oil-based drilling mud that includes a non-aqueous drilling fluid operable for use in the process of drilling boreholes, a wetting agent, a first emulsifier, and a second emulsifier. The wetting agent may be a mixture of polyamide imidazolines diluted with a solvent. The first emulsifier may be a fatty acid having a carbon number of twelve to fourteen. The second emulsifier may be a fatty acid with a carbon number of sixteen to eighteen. The drilling mud of this embodiment may contain a base oil, a viscosifier, water, calcium chloride, lime, a fluid loss additive, barite, and calcium carbonite. The drilling fluid of this embodiment may be free of tall oil and tall oil products.

### BRIEF DESCRIPTION OF THE DRAWINGS

FIG. 1: Infrared of Product M, with 1640 cm⁻¹ and 1609 cm⁻¹ corresponding to the C═O stretching frequencies in the amide and C═N imidazoline stretch, respectively.

While this disclosure is susceptible to various modifications and alternative forms, specific embodiments are shown by way of example in the drawings and will be described in detail. The drawings may not be to scale. It should be understood, however, that the drawings and the detailed descriptions thereto are not intended to limit the disclosure to the particular form disclosed, but, to the contrary, the intention is to cover all modifications, equivalents, and alternatives falling within the spirit and scope of the present disclosure as defined by the appended claims.

### DETAILED DESCRIPTION

Emulsifiers are an important part of a stable invert emulsion mud system. Common commercial mud systems contain a blend of a primary emulsifier and a secondary emulsifier (sometimes referred to as the wetting agent). In these systems, primary emulsifiers are typically comprised of tall oil fatty acid with or without mild chemical treatment. The primary emulsifiers form the basis for the emulsion. Invert emulsion systems also contain weighting agents which are materials used to give the mud systems additional density. Wetting agents improve the stability of the emulsion as well as to keep the weighting agents oil-wetted.

Many commercial emulsifiers used in mud systems use tall oil fatty acids (also known simply as “tall oil”) as the feedstock. Tall oil is a product of waste streams from paper milling and other processes and contains a complex mixture of components. Its exact composition varies depending on the wood used as the source. Typically it is derived from coniferous trees such as pine, cedars, junipers, and redwoods. As such, tall oil feedstocks are limited to areas where there is significant timber and paper production.

These complex mixtures typically can be further modified. For example, acid derivative reaction products include tall oil acids with polyamines such as diethylenetriamine (“DETA”), triethylenetetramine (“TETA”), or tetraethylenepentamine (“TEPA”). A further example includes the addition of maleic anhydride to increase performance to acceptable levels. Commercial examples of emulsifiers produced from modified tall oils include Halliburton Corp.'s (Houston, Tex., U.S.A.) Invermul® and Schlumberger Limited's (Houston, Tex., U.S.A.) Versamul®.

Regardless of the origin of the emulsifier, the rheological properties of drilling mud can be measured and compared in order to evaluate the emulsifier's effectiveness. The viscosity of the drilling fluid is kept within a range so that drill cuttings can be carried out of the wellbore. Drilling fluid performance can be tested using procedures described in API (American Petroleum Institute) Recommended Practice 13-B2 (5^{th} Edition, April 2014). These standards govern the testing of the drilling fluid's density, viscosity, gel strength, shear strength and other properties.

Embodiment emulsifiers do not depend on tall oil or its derivatives as a component. An improved method and product are disclosed. Instead of using tall oil fatty acids, synthetic linear saturated fatty acids are used to form embodiment emulsifiers. In some embodiments, different length chains of linear saturated fatty acid may be used such as C6-8, C12-14, or C16-18 as a starting point. The linear saturated fatty acids are heated to melting. C6-8 is a liquid at room temperature, while C12-14 has a melting point range of 44 to about 54° C. and C 16-18 has a melting point range of 63 to about 70° C. These fatty acids, alone or in combination, are reacted with polyamines, including the previously described DETA, TETA, and TEPA, and combinations thereof, to form embodiment emulsifiers. The fatty acid and the polyamine are mixed along with a catalytic amount of an acid, for example, 0.2% by weight of p-toluenesulfonic acid (“PTSA”), to form the described wetting agent. Various combinations of fatty acids and polyamines may be used.

### EXAMPLES

Formation of Embodiment Wetting Agents

Table 1 presents combinations of linear saturated fatty acids and polyamines with their ratios and provides a description of the solvent dilution. The molar ratio of the fatty acid to the polyamine in various embodiments is shown in Table 1. These reaction products are designed to function as the wetting agent in embodiment drilling fluids.

TABLE 1
Example Reaction Products from Various Combinations of Synthetic Linear Saturated
Fatty Acids and Polyamines with Diluents.

| Synthetic Linear | | Reaction | Reaction Product | Product |
|---|---|---|---|---|
| Fatty Acid Mixture | Polyamine | Ratio | Diluent | Designation |
| C6-8 | TEPA | 3:1 | None | A |
| C12-14 | DETA | 3:1 | 50% in EGBE | B |
| C12-14 | TETA | 2:1 | 50% in xylene | C |
| C12-14 | TEPA | 3:1 | 45% in xylene | D |
| C12-14 | TEPA | 4:1 | 50% in xylene | E |
| C16-18 | DETA | 2:1 | 45% in xylene | F |
| C16-18 | TEPA | 3:1 | 45% in xylene | G |
| C16-18 | TETA | 3:1 | 45% in xylene | H |
| C6-8 | TETA | 2:1 | 40% in xylene | I |
| C6-8 | DETA | 2:1 | 45% in xylene | J |
| C6-8 | TETA | 3:1 | 50% in xylene | K |
| C12-14 | TETA | 3:1 | 50% in xylene | L |
| C12-14 | TETA | 3:1 | 50% in 50:50 | M |
| | | | n-butanol: EGBE | |

For the embodiment products given in Table 1, the first reaction mixture of synthetic linear fatty acid mixture and polyamines at the molar ratio shown is heated at a first temperature for a first period of time (160° C. for four hours) until all of the water is driven off. The water-free first reaction products are collected in a Dean-Stark apparatus. A Dean-Stark apparatus is used here for illustrative purposes but one skilled in the art will recognize that any laboratory or commercial equipment may be used to separate the reaction products from water. This initial step creates a polyamide condensate through the formation of amides by reacting the linear saturated fatty acids with the polyamine. The water-free polyamide condensate mixture is then further heated at a second temperature for a second period (190° C. for 2-3 hours) to allow for the formation of an imidazoline species. The final product is a polyamide imidazoline, where one imidazoline moiety is present in the structure, with the remaining amine sites existing as amides in the structure.

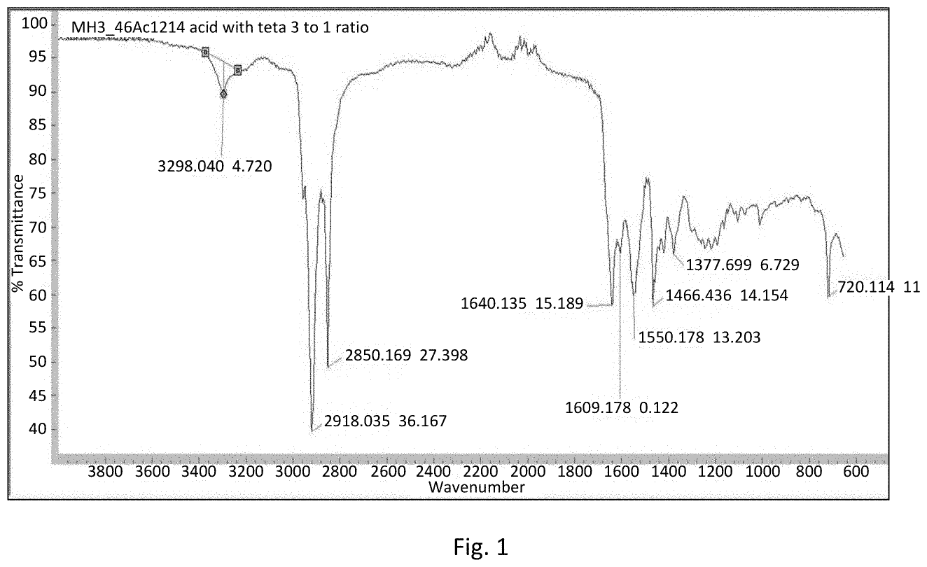

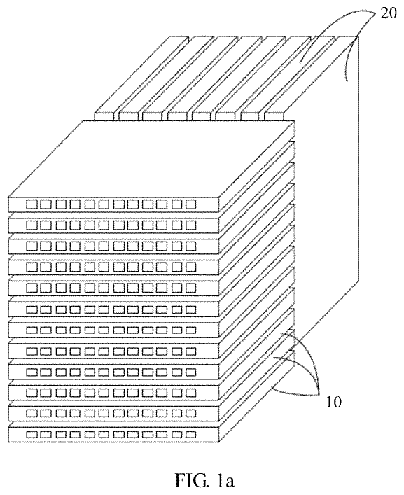

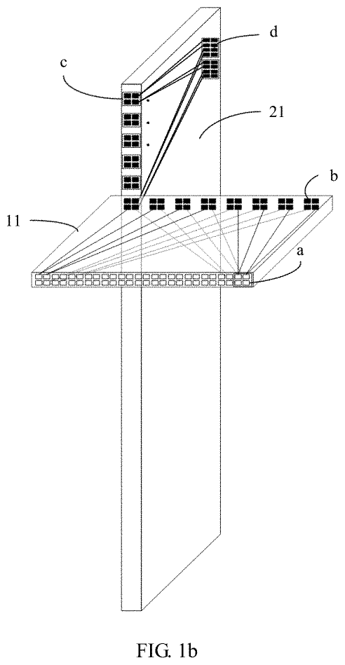

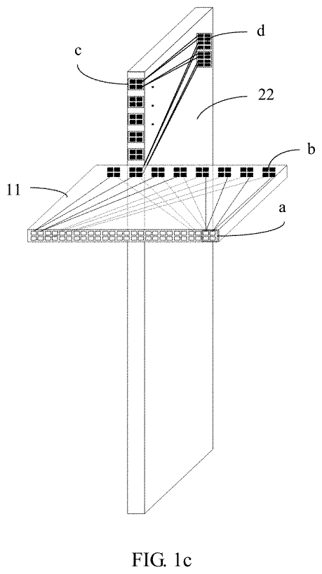

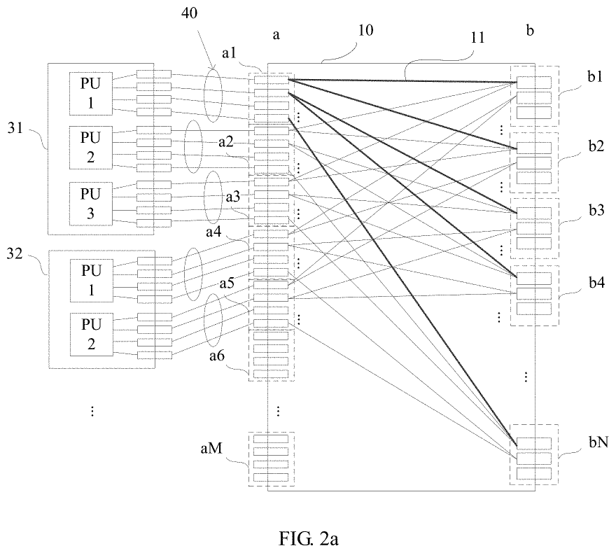

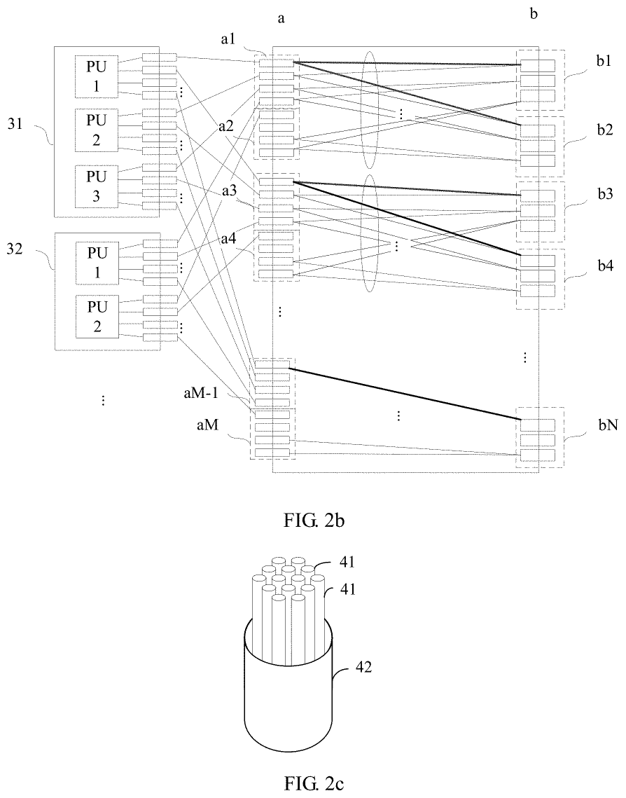

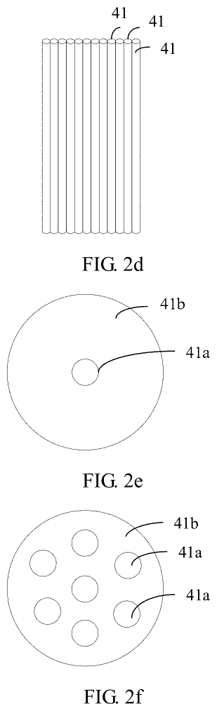

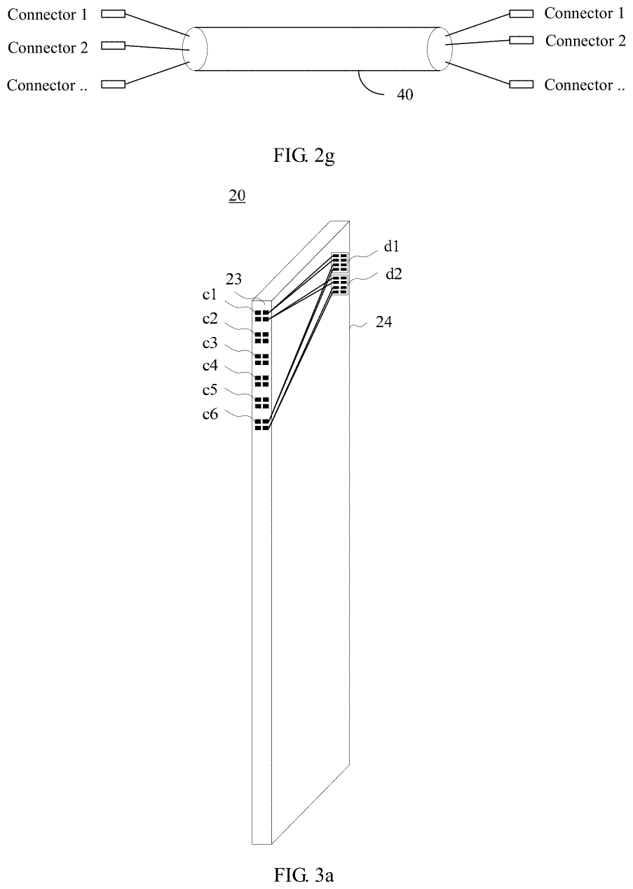

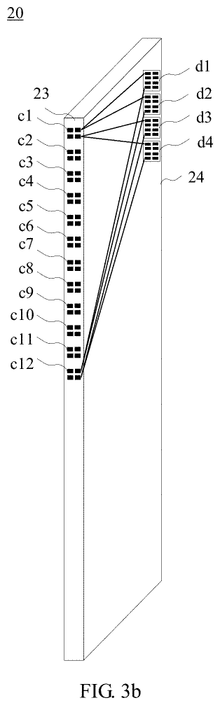

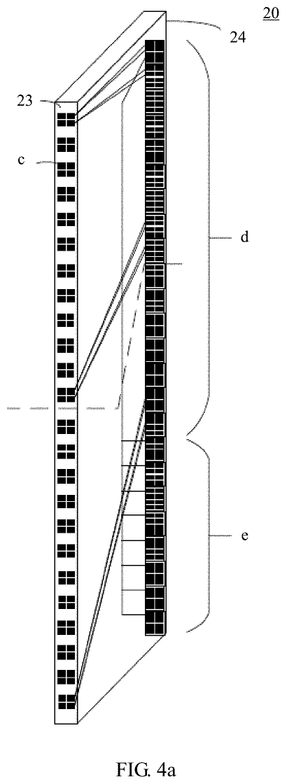

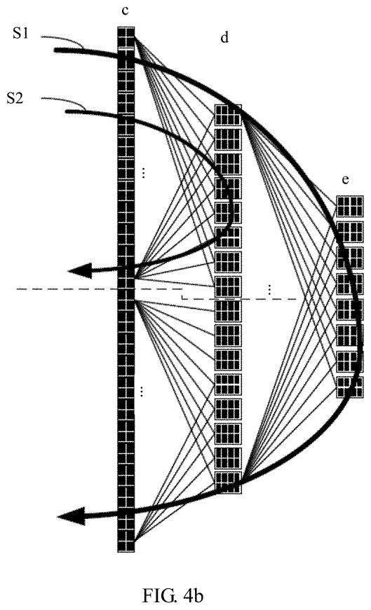

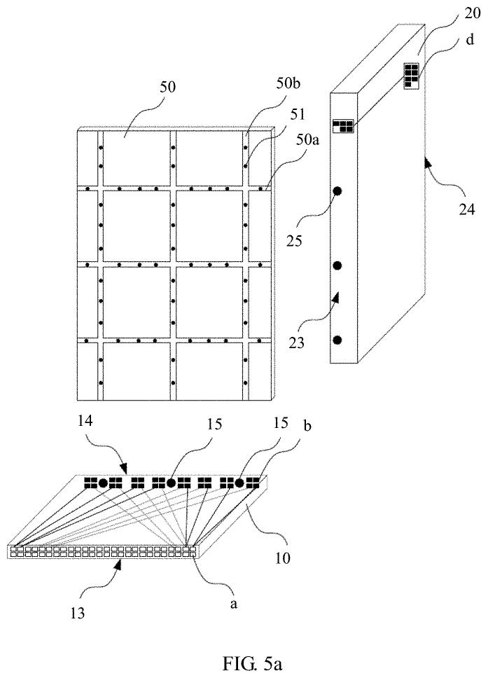

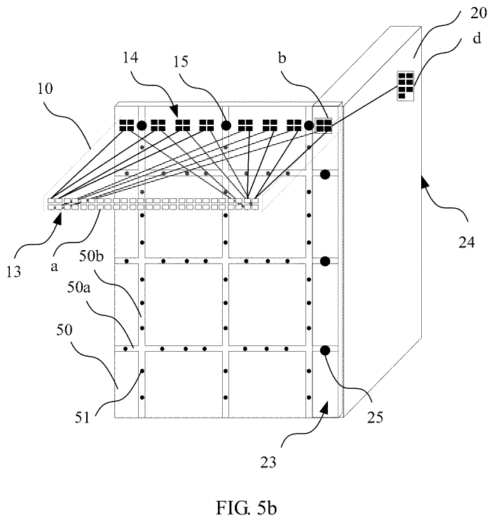

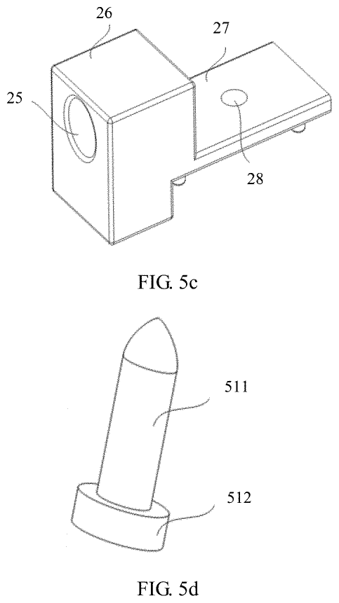

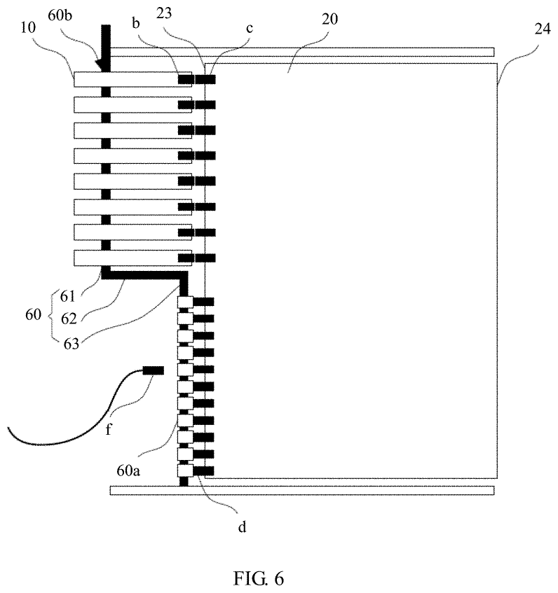

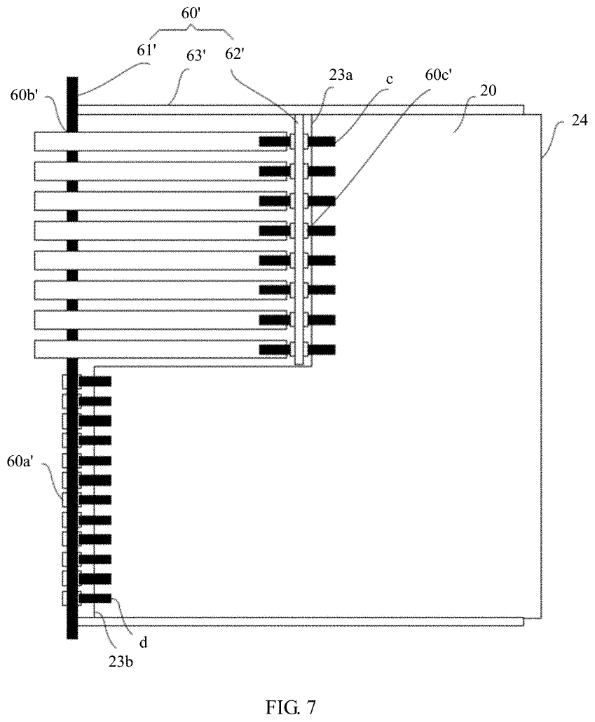

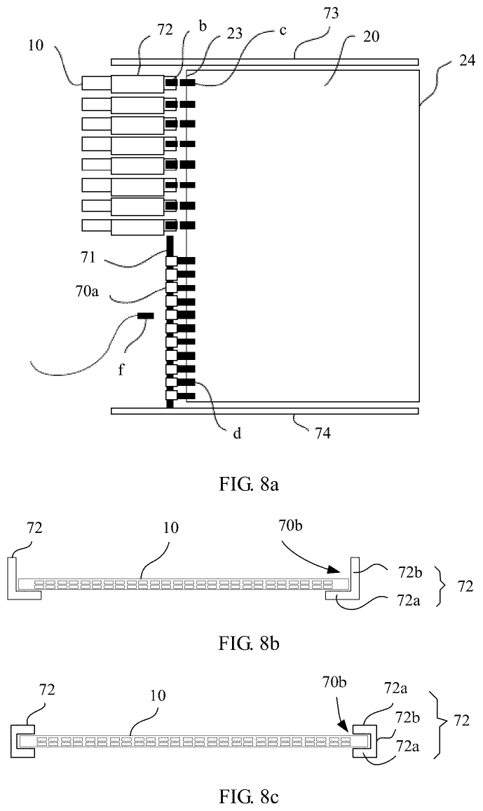

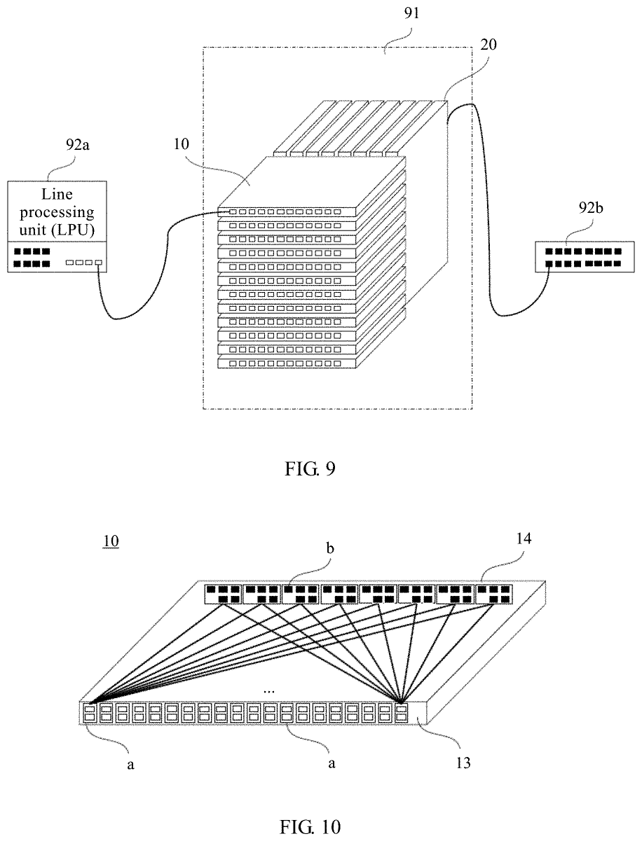

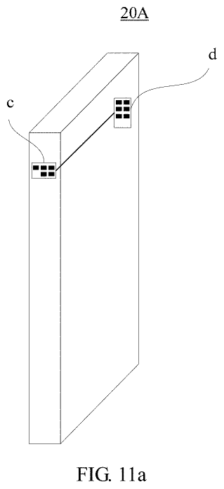

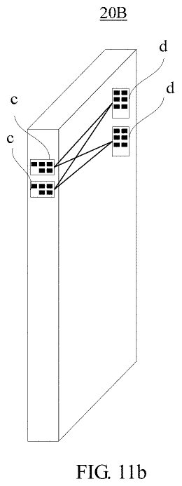

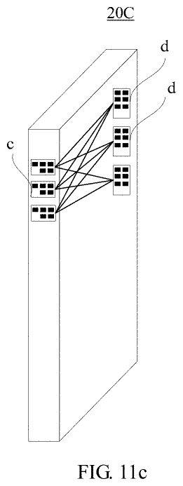

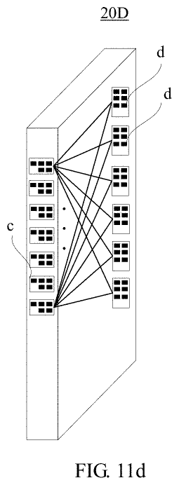

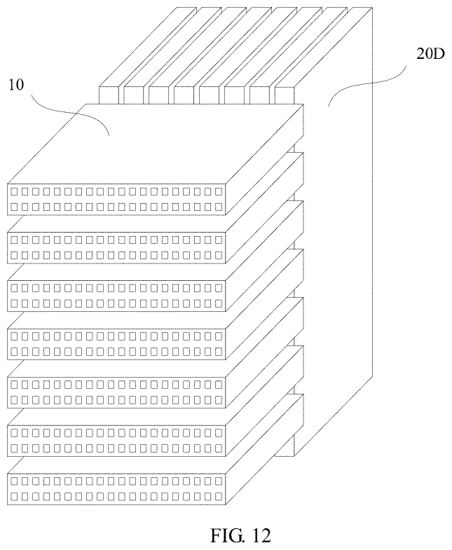

Formation of the amide-imidazoline species was confirmed by infrared spectroscopy. FIG. 1 included an infrared graph of Product M, with 1640 cm⁻¹ and 1609 cm⁻¹ peaks corresponding to the C═O stretching frequencies in the amide and C═N imidazoline stretch, respectively. The polyamide imidazoline species exhibits a waxy character at room temperature.

The polyamide imidazoline species is then further diluted with solvents such as 2-butoxyethanol/ethylene glycol butyl ether (“EGBE”), n-butanol, xylenes, or combinations thereof. The n-butanol/EGBE blend is able to provide a workable liquid at room temperature, whereas the xylene blends require elevated temperatures to flow. Example diluent and dilution percentages by mass are shown in Table 1. Although the results described, infra, are shown as diluted with EGBE or xylenes, in other embodiments, the solvent can be any solvent that accounts for the temperature needs of the final product. N-butanol and butyl cellosolve, and combinations thereof, are a common dilutent for emulsifiers and may be used in combination with the embodiment.

The embodiment products previously described may be used as a replacement for the wetting agent in standard mud formulations. Two embodiment mud formulations are presented in Tables 2 and 3. These mud formulations are presented as examples where Products A-M are substituted for standard commercial wetting agents. In other embodiments, the reaction products can be utilized in other mud formulations in addition to the formulations presented in Tables 2 and 3.

Tables 2 and 3 show example commercially available products to use in a drilling mud. Other commercially available products and additives known in the art may be substituted for the ones shown. For example, diesel is shown as the base oil, but other base oils such as isoparaffin, alpha-olefins, internal olefins, naphtha, kerosene, mineral oil, vegetable oil or others known in the art may be used in the mud formulation.

Table 2 presents the components of a mud formulation, including Geltone V®, Invermul®, and Duratone HT® obtained from Halliburton of Houston, Tex., U.S.A. In this formulation, the wetting agent is any of Products A-M or a commercial example (A) in the amount shown in the table.

TABLE 2
Example and Comparative Example
A Mud Formulations 1.

| | Units (lb/bbl unless |
|---|---|
| Component | otherwise noted) |
| Diesel | 0.58 bbl |
| Geltone V ® | 4 |
| Water | 0.18 bbl |
| CaCl₂ | 45 |
| Lime | 6 |
| Invermul ® | 12 |
| Product A-M or | 6 |
| Commercial Ex. A | |
| Duratone HT ® | 7 |
| Barite | 120 |
| CaCO₃ fine | 5 |

Table 3 presents the components of a mud formulation, including VG-69®, Versamul®, Asphasol®, Safecarb® 10, and Safecarb® 40 obtained from Schlumberger Ltd. of Houston, Tex., U.S.A. and Duratone HT® obtained from Halliburton Corp. of Houston, Tex., U.S.A. Similar to Table 2, in the formulation shown in Table 3, the wetting agent is any of Products A-M or a commercial example (B).

TABLE 3
Example and Comparative Example
B Mud Formulations 2.

| | Units (lb/bbl unless |
|---|---|
| Component | otherwise noted) |
| Diesel | 0.58 bbl |
| VG-69 ® | 8 |
| Lime | 5 |
| Versamul ® | 12.5 |
| Product A-M or | 6.2 |
| Commercial Ex. B | |
| Water | 0.18 bbl |
| CaCl₂ | 17 |
| Duratone HT ® | 7 |
| Asphasol ® | 7 |
| RM 63 | 0.7 |
| Safecarb ® 10 | 15 |
| Safecarb ® 40 | 15 |
| Barite | 172 |

The Products A-M as shown in Table 1 were used to replace the wetting agents in mud formulations shown in Tables 2 and 3. The testing results are presented in Tables 7 and 8. The drilling fluids were prepared and tested using testing procedures described in API (American Petroleum Institute) Recommended Practice 13-B2 (5^{th} Edition, April 2014). The tests were conducted at high pressure (10,000 psi) and high temperature (150° F.-400° F.) downhole conditions. Plastic viscosity (PV), yield point (YP), apparent viscosity (AV), low shear yield point (LSYP), and gel strength at 10 seconds and 10 minutes were all measured and recorded.

TABLE 7
Rheological Test Results for Mud Formulation 1
(Fann 35 data, 150° F. and atmospheric pressure)

| | Fann Dial Reading | | | | | | Gel Strength | | | | | |
|---|---|---|---|---|---|---|---|---|---|---|---|---|
| | (rpm) | | | | | | (lb/100 ft²) | | Rheology | | | |
| Wetting | 600 | 300 | 200 | 100 | 6 | 3 | 10 | 10 | PV | YP | AV | LSYP |
| Agent | rpm | rpm | rpm | rpm | rpm | rpm | s | m | cp | lb/100 ft² | cp | lb/100 ft² |
| Commercial | 30 | 18 | 13 | 9 | 3 | 3 | 3 | 5 | 12 | 6 | 15 | 3 |
| Example A | | | | | | | | | | | | |
| Product A | 33 | 19 | 14 | 9 | 4 | 4 | 5 | 5 | 14 | 5 | 16.5 | 4 |
| Product B | 32 | 18 | 13 | 8 | 2 | 2 | 3 | 5 | 14 | 4 | 16 | 2 |
| Product C | 32 | 18 | 13 | 8 | 3 | 3 | 4 | 5 | 14 | 4 | 16 | 3 |
| Product D | 30 | 18 | 13 | 9 | 3 | 3 | 5 | 6 | 12 | 6 | 15 | 3 |
| Product E | 34 | 21 | 16 | 10 | 5 | 4 | 5 | 7 | 13 | 8 | 17 | 3 |
| Product F | 29 | 17 | 12 | 8 | 3 | 3 | 3 | 5 | 12 | 5 | 14.5 | 3 |
| Product G | 32 | 18 | 13 | 9 | 3 | 3 | 4 | 6 | 14 | 4 | 16 | 3 |
| Product H | 34 | 21 | 15 | 10 | 4 | 4 | 5 | 6 | 13 | 8 | 17 | 4 |

TABLE 8
Rheological Test Results for Mud Formulation 2
(Fann 35 data, 150° F. and atmospheric pressure)

| | Fann Dial Reading | | | | | | Gel Strength | | | | | |
|---|---|---|---|---|---|---|---|---|---|---|---|---|
| | (rpm) | | | | | | (lb/100 ft²) | | Rheology | | | |
| Wetting | 600 | 300 | 200 | 100 | 6 | 3 | 10 | 10 | PV | YP | AV | LSYP |
| Agent | rpm | rpm | rpm | rpm | rpm | rpm | s | m | cp | lb/100 ft² | cp | lb/100 ft² |
| Commercial | 57 | 35 | 24 | 16 | 7 | 8 | 10 | 21 | 22 | 13 | 8 | 12 |
| Example B | | | | | | | | | | | | |
| Product A | 51 | 30 | 20 | 14 | 9 | 10 | 14 | 16 | 21 | 9 | 7 | 18 |
| Product B | 60 | 34 | 24 | 16 | 8 | 8 | 11 | 22 | 26 | 8 | 8 | 14 |
| Product C | 71 | 42 | 26 | 17 | 9 | 9 | 12 | 19 | 29 | 13 | 8.5 | 15 |
| Product D | 59 | 36 | 28 | 20 | 12 | 12 | 17 | 33 | 23 | 13 | 10 | 22 |
| Product E | 58 | 35 | 26 | 19 | 12 | 12 | 15 | 26 | 23 | 12 | 9.5 | 18 |
| Product F | 60 | 38 | 30 | 21 | 14 | 14 | 18 | 27 | 22 | 16 | 10.5 | 22 |
| Product G | 60 | 36 | 28 | 20 | 11 | 12 | 15 | 24 | 24 | 12 | 10 | 18 |
| Product H | 75 | 45 | 35 | 25 | 15 | 15 | 22 | 32 | 30 | 15 | 37.5 | 15 |

The Products outperformed the incumbents in tests. As shown in Tables 7 and 8, the Products A-M show equivalent or improved performance over both Commercial Example A (Table 7) and Commercial Example B (Table 8) at 150° F. and ambient pressure including improving low shear rheological properties. For example, Product H has similar PV to the commercial examples, but improved (that is, greater) YP and LSYP. Table 7 presents results based upon the mud formulation described in Table 2 while Table 8 presents results based upon the mud formulation described in Table 3.

High Pressure/High Temperature Drilling Fluid Tests

Due to the importance of high temperature, high pressure (HTHP) drilling, additional testing was performed on Products D and L at high temperature (150° F.-400° F.) and pressure (10,000 psi). The Products were compared to Commercial Example A in Mud Formulation 1 (shown if Table 2).

TABLE 8
Yield Point (YP) comparison at 10,000 PSI.

| Temp. | Commercial | | |
|---|---|---|---|
| (°F) | Example A | Product D | Product L |
| 150 | 12 | 17 | 20 |
| 200 | 7 | 12 | 15 |
| 250 | 6 | 6 | 13 |
| 300 | 5 | 6 | 10 |
| 350 | 6 | 5 | 10 |
| 400 | 2 | 6 | 7 |

TABLE 10
Low Shear Yield Point (LSYP) comparison at 10,000 PSI.

| Temp. | Commercial | | |
|---|---|---|---|
| (°F) | Example A | Product D | Product L |
| 150 | 4 | 7 | 7 |
| 200 | 5 | 5 | 6 |
| 250 | 3 | 6 | 7 |
| 300 | 3 | 5 | 7 |
| 350 | 5 | 5 | 7 |
| 400 | 4 | 4 | 6 |

The results, as set forth in Table 9 and 10, showed distinct improved performance of the reaction products versus the incumbents. At these elevated temperatures and pressures, the fluid should exhibit low friction potential, and a stable yield point and low shear rheological values to prevent barite settling and reduce stuck pipe and maintain wellbore stability. For both YP (Table 9) and LSYP (Table 10), both Products D and L maintain a stable range as compared to Commercial Example A, especially at elevated temperatures such as 400° F. At this elevated temperature, the YP and LSYP of the Commercial Examples declines more than the Products.

Synthetic Linear Fatty Acid and Wetting Agent System Examples

In other embodiments, drilling muds may be produced where the Products A-M listed in Table 1 are used as the wetting agent and the primary emulsifier is replaced with synthetic fatty acids such as C12-14 and C-16-18 either alone or in combination. Synthetic fatty acids are available from major chemical companies. Example mud formulations using the full emulsifier package replacement formulation described previously are presented in Tables 4 and 5. A comparative mud system using a primary emulsifier produced from tall oil feedstocks is shown in Table 6.

In addition to the comparison of wetting agents (secondary emulsifiers), in some embodiments the industry primary emulsifiers were replaced with the synthetic fatty acids C12-14, C16-18. The mud systems shown in Tables 4 and 5 are formulations that show a full emulsifier package replacement. A commercial mud system is shown in Table 6.

TABLE 4
Example Mud Formulation with Full Emulsifier
Replacement (Mud System 1).

| | Units (lb/bbl unless |
|---|---|
| Component | otherwise noted) |
| Diesel) | 0.58 bbl |
| Geltone V ® | 4 |
| Water | 0.18 bbl |
| CaCl₂ | 45 |
| Lime | 6 |
| SABIC C12-14 fatty | 6 |
| acid mixture | |
| SABIC C16-18 fatty | 6 |
| acid mixture | |
| Product L | 6 |
| Duratone HT ® | 7 |
| Barite | 120 |
| CaCO₃ fine | 5 |

TABLE 5
Example Mud Formulation with Full Emulsifier
Replacement (Mud System 2).

| | Units (lb/bbl unless |
|---|---|
| Component | otherwise noted) |
| Diesel | 0.58 bbl |
| Geltone V ® | 4 |
| Water | 0.18 bbl |
| CaCl₂ | 45 |
| Lime | 6 |
| SABIC C12-14 fatty | 4 |
| acid mixture | |
| SABIC C16-18 fatty | 4 |
| acid mixture | |
| Product L | 10 |
| Duratone HT ® | 7 |
| Barite | 120 |
| CaCO₃ fine | 5 |

TABLE 6
Comparative Example Mud System
(Comparative Example).

| | Units (lb/bbl unless |
|---|---|
| Component | otherwise noted) |
| Diesel | 0.58 bbl |
| Geltone V ® | 4 |
| Water | 0.18 bbl |
| CaCl₂ | 45 |
| Lime | 6 |
| Primary Emulsifier | 12 |
| Commercial Ex. A | 6 |
| Duratone HT ® | 7 |
| Barite | 120 |
| CaCO₃ fine | 5 |

In Tables 3-6, the Geltone V® and Duratone HT® were obtained from Halliburton of Houston, Tex., U.S.A. The SABIC C12-14 fatty acid mixture and the SABIC C16-18 fatty acid mixture were obtained from Saudi Arabia Basic Industries Corp. (SABIC) of Riyadh, Kingdom of Saudi Arabia.

The two new mud systems showed improvement in several tests over the commercial mud system. See Table 11.

TABLE 11
Plastic Viscosity Comparison of the Commercial Formulation and
Mud Systems 1 and 2 at 10,000 PSI.

| Temp. | Commercial | Mud | Mud |
|---|---|---|---|
| (°F) | Formulation | System 1 | System 2 |
| 150 | 23 | 37 | 45 |
| 200 | 15 | 14 | 14 |
| 250 | 11 | 9 | 8 |
| 300 | 9 | 7 | 6 |
| 350 | 7 | 5 | 4 |
| 400 | 8 | 3 | 2 |

For both mud systems, PV, an indicator of the friction force required to move the fluid, decreased as the temperature increased, whereas the commercial mud system exhibited greater PV at high temperatures as shown in Table 11. A low PV is an advantage since higher temperatures are observed in deeper well sections. Any increase in friction forces directly contributes to the force required to move the fluid. Increased friction force can increase the pressure applied to the formation such that the actual pressure passes past the point of fracture, thereby inducing lost zones and wellbore instability. A lower PV reduces friction forces.

As drilling fluids increase in temperature, the rheological properties decrease. An improved fluid system will decrease more slowly or remain stable at higher temperatures. Table 12 demonstrates the change in YP as a function of temperature for the two mud systems and the commercial formulation.

TABLE 12
Yield Point (YP) comparison of the Commercial Example and
Mud Systems 1 and 2 at 10,000 PSI.

| Temp. | Commercial | Mud | Mud |
|---|---|---|---|
| (°F) | Formulation | System 1 | System 2 |
| 150 | 12 | 24 | 30 |
| 200 | 7 | 14 | 20 |
| 250 | 6 | 10 | 14 |
| 300 | 5 | 8 | 10 |
| 350 | 6 | 8 | 9 |
| 400 | 2 | 8 | 9 |

YP correlates to the ability of the fluid to clean the hole, carry cuttings, and keep the weighting agent suspended for proper density control. The YP of the product is improved over the incumbent over all temperature ranges, especially at 400° F. where the YP of the commercial example decreases. This decrease indicates that the fluid system at 400° F. is not stable. LSYP exhibits a similar trend as shown in Table 13, where at or less than a value of 5 is known to demonstrate a fluid is has reduced stability.

TABLE 13
Low Shear Yield Point (LSYP) comparison of the Commercial
Example and Mud Systems 1 and 2 at 10,000 PSI.

| Temp. | Commercial | Mud | Mud |
|---|---|---|---|
| (°F) | Formulation | System 1 | System 2 |
| 150 | 4 | 11 | 15 |
| 200 | 5 | 6 | 7 |
| 250 | 3 | 5 | 6 |
| 300 | 3 | 6 | 7 |
| 350 | 5 | 6 | 6 |
| 400 | 4 | 6 | 6 |

The synergy between the synthetic linear saturated acids, and the condensates described based on synthetic linear saturated acids leads to a higher temperature performance over commercial examples utilize natural product streams such as tall oil.

Additional Example Polyamide and Polyamide Imidazolines Emulsifiers

Embodiments of the disclosure further include polyamides having the chemical formulas described infra. Embodiments of the disclosure may also include polyamide imidazolines produced by dehydration of the polyamides, as also described infra.

Embodiments of the disclosure include a process for manufacturing the polyamides and the polyamide imidazolines from a polyamine and one or more saturated fatty acids. In such embodiments, the polyamine may have the following formula:

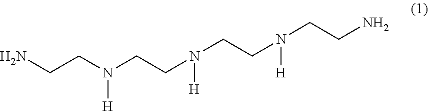 [SMILES: NCCNCCNCCNCCN]

In some embodiments, the polyamine is triethylenetetramine (TETA).

In such embodiments, the one or more saturated fatty acids have the following structure:

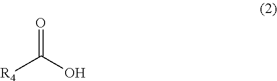

Where R₄ is selected from the group consisting of C₁₁H₂₃, C₁₂H₂₅, and C₁₃H₂₇.

The polyamides (referred to as Polyamide-1 and Polyamide-2) produced from the reaction of the polyamine shown in Formula 1 and the linear saturated fatty acid shown in Formula 2 may have the following formulas:

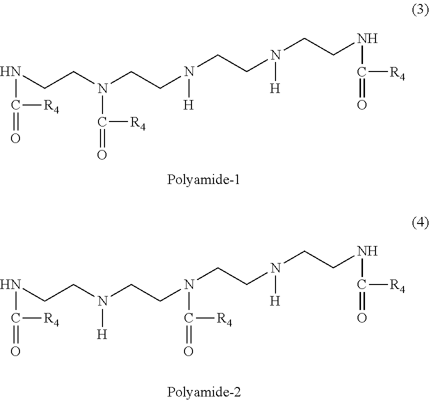

Where R₄ is selected from the group consisting of C₁₁H₂₃, C₁₂H₂₅, and C₁₃H₂₇.

The mixture of polyamide imidazolines produced by dehydration of a condensate of the polyamides shown in Formulas 3 and 4 may have one end cyclized or both ends cyclized. The mixture of polyamide imidazolines produced by dehydration of the polyamide condensate shown in Formulas 3 and 4 may have the following formulas:

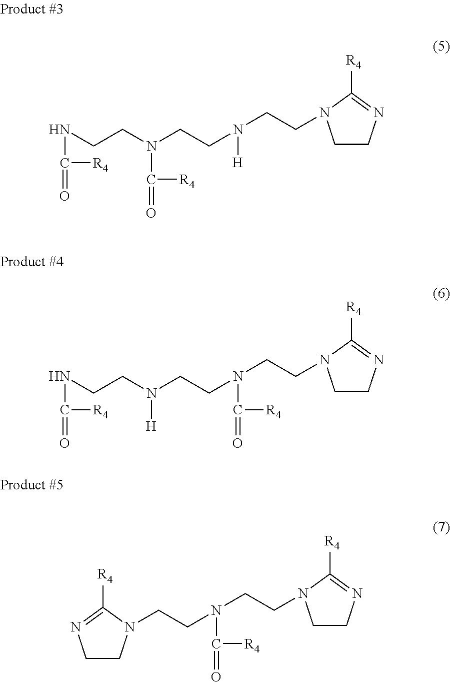

Where R₄ is selected from the group consisting of C₁₁H₂₃, C₁₂H₂₅, and C₁₃H₂₇

Embodiments further include a process for synthesizing the polyamides and polyamide imidazolines from the polyamine shown in Formula 1 and the saturated fatty acid shown in Formula 2. In some embodiments, the process includes the following steps:

1) Preheat the saturated fatty acids to a temperature of at least 85° C.

2) Preheat the reactor to a temperature of at least 100° C. while blanketing with nitrogen (N₂), engage an agitator, add measured amount of the preheated saturated fatty acid to reactor, and leave the reflux condenser open to remove formed water.

3) Add measured amount acid (for example, p-tolenesulfonic acid (PTSA) monohydrate) to reactor.

4) Increase the reactor temperature to at least 130° C.

5) Gradually add the polyamine into the reactor.

6) After the initial exothermic reaction, heat the reactor to at least 160° C. for a time period of at least 4 hours and monitor the amount of water collected in the reflux condenser.

7) Compare the amount of collected water to the theoretical amount of water to be removed.

8) When the evaporation of water has ceased, heat the reactor to at least 180° C. and continue to leave the reflux condenser open.

9) Obtain periodic (for example, hourly) samples and test for the formation of polyamide imidazoline products via infrared spectroscopy. In some embodiments, the polyamide imidazoline products may show peaks at 1550 cm⁻¹, 1607 cm⁻¹, 1640 cm⁻¹, or combinations thereof. The reaction may continue until the IR spectrum no longer changes, at which point the synthesis may be considered complete.

In some embodiments, the molar ratio of the linear saturated fatty acid shown in Formula 2 to the polyamine shown in Formula 1 may be in the range of 2:1 to 4:1. In some embodiments, the molar ratio of the saturated fatty acid shown in Formula 2 to the polyamine shown in Formula 1 may be 3:1. In some embodiments, the acid (for example, PTSA) may be 0.2% by weight of the saturated fatty acid and polyamine.

In some embodiments, the reaction mixture may include a solvent. The solvent may include xylene, ethylene glycol butyl ether, n-butanol, or any combination thereof. Additionally or alternatively, the solvent may include butyl Cellosolve™ manufactured by Dow Inc. of Midland, Mich., USA.

In one example embodiment, polyamide imidazolines were produced from 48.84 grams (g) of the linear saturated fatty acids shown in Formula 2 and the polyamine shown in Formula 1. The reaction components and corresponding amounts are shown in Table 14:

TABLE 14
Reaction Components and Amounts for Synthesis of
Polyamide Emulsifiers and Polyamide Imidazolines

| Component | Weight (g) |
|---|---|
| Linear saturated fatty acids | 48.84 |
| Polyamine | 11.1 |
| PTSA | 0.06 |
| N-Butanol | 20 |
| Co-solvent (butyl Cellosolve ™) | 20 |

The theoretical weight of the reaction product from the components in Table 14 was estimated at 55.83 g. The actual weight of the reaction product was 55.3 g.

In some embodiments, an oil-based drilling mud includes a drilling fluid and a wetting agent having the mixture of polyamide imidazolines selected from the group shown in Formulas 5-7. In such embodiments, non-aqueous drilling fluid may include a base oil, a viscosifier, water, calcium chloride (CaCl₂), lime (Ca(OH)₂), an emulsifier, a fluid loss additive, barite, and calcium carbonate (CaCO₃), as described supra. In some embodiments, the oil-based drilling mud may have the formulation shown in Table 2 or Table 3, with the mixture of polyamide imidazolines selected from the group shown in Formulas 5-7 used as the wetting agent.

In some embodiments, an oil-based drilling mud that includes a drilling fluid and a wetting agent having the mixture of polyamide imidazolines selected from the group shown in Formulas 5-7 may also include a synthetic fatty acid as the emulsifier. Such fatty acids may include C12-14 fatty acids, C16-18 fatty acids, and any combination thereof. In such embodiments, the oil-based drilling mud may have the formulation shown in Table 4 or Table 5, with the mixture of polyamide imidazolines selected from the group shown in Formulas 5-7 used as the wetting agent.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the embodiments. It is to be understood that the forms of the embodiments shown and described are to be taken as examples of embodiments. Elements and materials may be substituted for those illustrated and described, parts and processes may be reversed or omitted, and certain features of the embodiments may be utilized independently, all as would be apparent to one skilled in the art after having the benefit of this description of the embodiments. Changes may be made in the elements described without departing from the spirit and scope of the embodiments as described in the following claims. Headings are for organizational purposes only and are not meant to be used to limit the scope of the description.

As used throughout this application, the word “may” is used in a permissive sense (that is, meaning having the potential to), rather than the mandatory sense (that is, meaning must). The words “include,” “including,” and “includes” mean including, but not limited to. As used throughout this application, the singular forms “a”, “an,” and “the” include plural referents unless the content clearly indicates otherwise. Thus, for example, reference to “an element” may include a combination of two or more elements. As used throughout this application, the term “from” does not limit the associated operation to being directly from. Thus, for example, receiving an item “from” an entity may include receiving an item directly from the entity or indirectly from the entity (for example, via an intermediary entity).

As used, the words “comprise,” “has,” “includes”, and all other grammatical variations are each intended to have an open, non-limiting meaning that does not exclude additional elements, components or steps. Embodiments of the present invention may suitably “comprise”, “consist” or “consist essentially of” the limiting features disclosed, and may be practiced in the absence of a limiting feature not disclosed. Thus, “comprising” includes “consisting essentially of” and “consisting of.”

What is claimed is:

1. A wetting agent composition comprising:
mixed polyamide imidazolines, wherein the mixed polyamide imidazolines are selected from the group consisting of: 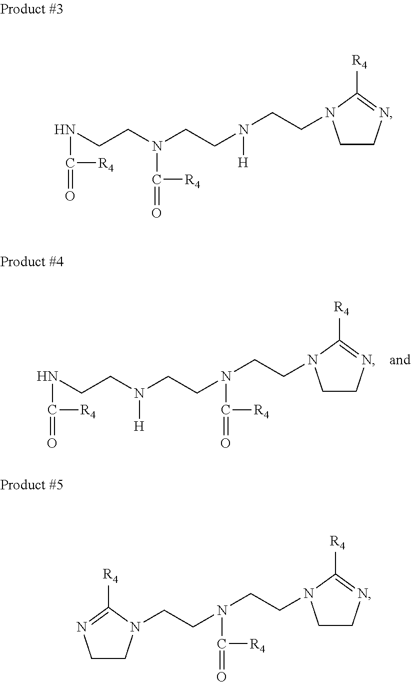
where R₄ is selected from the group consisting of C₁₁H₂₃, C₁₂H₂₅, and C₁₃H₂₇.

2. The wetting agent composition of claim 1, wherein the mixed polyamide imidazolines are prepared by contacting mixed saturated fatty acids with a polyamine in the presence of an acid, forming a polyamide condensate at a first set of conditions, eliminating water from the presence of the polyamide condensate, and forming the mixed polyamide imidazolines at a second set of conditions, wherein the mixed saturated fatty acids comprise: 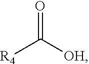
where R₄ is selected from the group consisting of C₁₁H₂₃, C₁₂H₂₅, and C₁₃H₂₇, and the polyamine comprises: 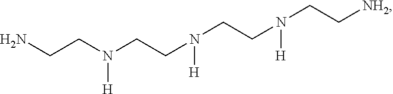
where R₄ is selected from the group consisting of C₁₁H₂₃, C₁₂H₂₅, and C₁₃H₂₇.

3. The wetting agent composition of claim 2, wherein the polyamide is selected from the group consisting of: 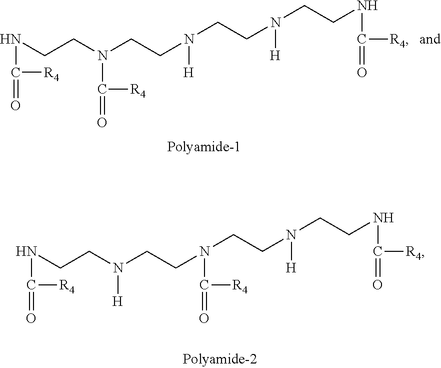
where R₄ is selected from the group consisting of C₁₁H₂₃, C₁₂H₂₅, and C₁₃H₂₇.

4. The wetting agent composition of claim 2 where the mixed saturated fatty acids and the polyamine are contacted in a molar ratio in a range of 2:1 to 4:1.

5. The wetting agent composition of claim 2 where the acid comprises p-toluenesulfonic acid (PTSA).

6. The wetting agent composition of claim 2 where the first set of conditions include a reaction temperature of at least 160° C. and a time period of at least 4 hours.

7. The wetting agent composition of claim 1 where the first set of conditions include a reaction temperature at least 180° C.

8. A method for the preparation of a wetting agent for use with a non-aqueous drilling fluid to create an altered oil-based drilling mud, comprising the steps of:
a) mixing a linear saturated fatty acid with a polyamine in the presence of p-toluenesulfonic acid (PTSA);
b) heating the mixture to a first temperature;
c) maintaining the mixture at the first temperature for a first time period to create a first reaction product;
d) heating the mixture to a second temperature greater than the first temperature;
e) maintaining the mixture at the second temperature for a second time period to create a second reaction product, wherein the second reaction product comprises mixed polyamide imidazolines, wherein the mixed polyamide imidazolines are selected from the group consisting of: 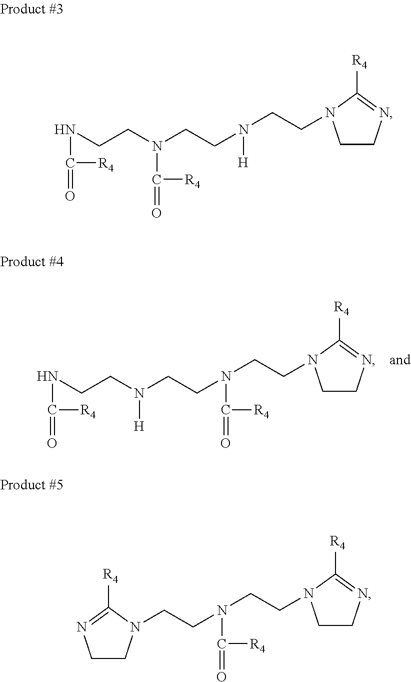
where R₄ is selected from the group consisting of C₁₁H₂₃, C₁₂H₂₅, and C₁₃H₂₇.

9. The method of claim 8, wherein the first time period is four hours.

10. The method of claim 8, wherein the mixed saturated fatty acids comprise: 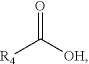
where R₄ is selected from the group consisting of C₁₁H₂₃, C₁₂H₂₅, and C₁₃H₂₇, and the polyamine comprises: 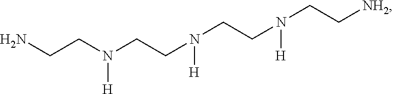
where R₄ is selected from the group consisting of C₁₁H₂₃, C₁₂H₂₅, and C₁₃H₂₇.

11. The method of claim 8, wherein the first reaction product comprises a polyamide condensate, wherein the polyamide is selected from the group consisting of: 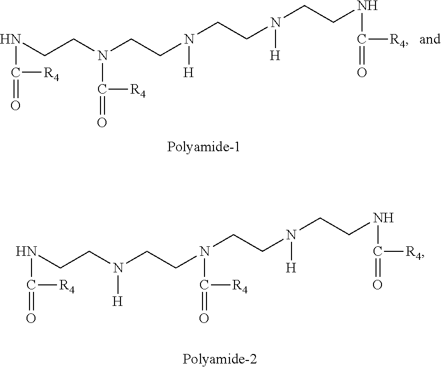
where R₄ is selected from the group consisting of C₁₁H₂₃, C₁₂H₂₅, and C₁₃H₂₇.

12. The method of claim 8, wherein the second time period is in the range of 2 to 3 hours.

13. The method of claim 8, wherein the amount of p-toluenesulfonic acid is 0.2% by weight.

14. The method of claim 8, wherein the second reaction product is diluted with a solvent.

15. The method of claim 8, wherein the solvent is selected from the group consisting of xylene, ethylene glycol butyl ether, and n-butanol.

16. The method of claim 8, wherein the first temperature is 160° C.

17. The method of claim 8, wherein the second temperature is 180° C.

18. An oil-based drilling mud comprising:
a non-aqueous drilling fluid; and
a wetting agent, the wetting agent comprising mixed polyamide imidazolines,
wherein the mixed polyamide imidazolines are selected from the group consisting of: 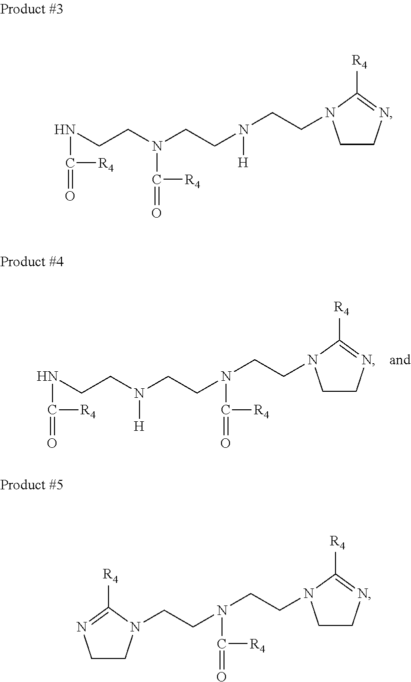
where R₄ is selected from the group consisting of C₁₁H₂₃, C₁₂H₂₅, and C₁₃H₂₇.

19. The oil-based drilling mud of claim 18, wherein the non-aqueous drilling fluid comprises:
base oil;
a viscosifier;
water;
calcium chloride;
lime;
an emulsifier;
a fluid loss additive;
barite; and
calcium carbonite.

20. The oil-based drilling mud of claim 18, wherein the mixed polyamide imidazolines are prepared by contacting mixed saturated fatty acids with a polyamine in the presence of an acid, forming a polyamide condensate at a first set of conditions, eliminating water from the presence of the polyamide condensate, and forming the mixed polyamide imidazolines at a second set of conditions, wherein the mixed saturated fatty acids comprise: 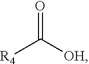
where R₄ is selected from the group consisting of C₁₁H₂₃, C₁₂H₂₅, and C₁₃H₂₇, and the polyamine comprises: 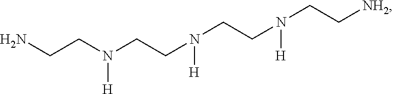
where R₄ is selected from the group consisting of C₁₁H₂₃, C₁₂H₂₅, and C₁₃H₂₇.

21. The oil-based drilling mud of claim 18, wherein the polyamide is selected from the group consisting of: 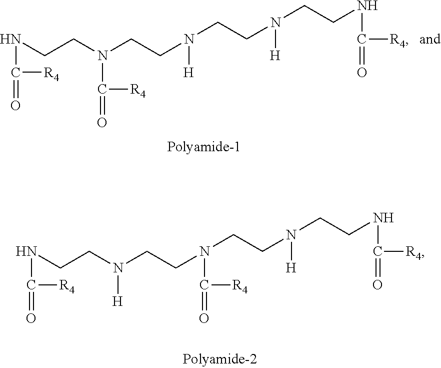
where R₄ is selected from the group consisting of C₁₁H₂₃, C₁₂H₂₅, and C₁₃H₂₇.
